(19) United States
(12) Patent Application Publication
(10) Pub. No.: US 2020/0289425 A1
(43) Pub. Date: Sep. 17, 2020

(54) CARBON DOT LIPOSOMES AND USES THEREOF

(71) Applicant: National Taiwan University, Taipei (TW)

(72) Inventors: Huan-Tsung CHANG, Taipei City (TW); Yu-Feng LIN, Kaohsiung City (TW); Shih-Chun WEI, New Taipei City (TW); Yu-Ting TSENG, New Taipei City (TW); Yu-Feng HUANG, Hsinchu City (TW); Chih-Ching HUANG, Keelung City (TW); Yu-Syuan LIN, Kaohsiung City (TW); Tzu-Heng CHEN, Hsinchu City (TW)

(73) Assignee: National Taiwan University, Taipei (TW)

(21) Appl. No.: 16/892,272

(22) Filed: Jun. 3, 2020

Related U.S. Application Data
(63) Continuation-in-part of application No. 15/948,550, filed on Apr. 9, 2018, now Pat. No. 10,702,483.
Application No. 15/948,550 is a continuation of application No. 15/440,353, filed on Feb. 23, 2017, now Pat. No. 9,962,337.

(51) Int. Cl. A61K 9/51; A61K 9/06

(52) U.S. Cl. CPC A61K 9/51; A61K 9/06; A61K 9/5123

(57) ABSTRACT

Disclosed herein are drug-containing vesicles, each of which includes a carbon dot liposome (C-dot liposome) formed by a plurality of Janus particles, which are self-assembled into the C-dot liposome; and a drug encapsulated within the C-dot liposome. Also disclosed herein is a method of producing the drug-containing vesicles. The method includes, mixing a plurality of Janus particles with a drug solution (e.g., an anti-cancer drug solution) to form a mixed solution; and producing the drug-containing vesicles either by a film-hydration method or an injection method. In the film-hydration method, the mixed solution is condensed until a film-like structure is formed; and sonicating the film-like structure in a salt solution to produce the drug-containing vesicle. In the injection method, the mixed solution is rapidly injected into a salt solution to produce the drug-containing vesicle. Also encompasses in the present disclosure are methods for treating a subject afflicted with a cancer. In some embodiments, the method includes administering an effective amount of the drug-containing vesicles to the subject to suppress the growth of the cancer. In other embodiments, the method includes administering an effective amount of C-dot liposome to the subject; and irradiating the subject with a first and a second wavelength of 350-400 nm and 480-550 nm.

### CROSS REFERENCE TO RELATED APPLICATIONS

This application is a continuation-in-part of pending U.S. Ser. No. 15/948,550 filed Apr 09, 2018, which is a continued application of U.S. Ser. No. 15/440,353 filed Feb 23, 2017; the disclosure of afore-indicated prior applications are incorporated herein by reference in their entireties.

### BACKGROUND OF THE INVENTION

### 1. Field of the Invention

The present disclosure in general relates to carbon dot liposomes and their uses for the treatment of a cancer.

### 2. Description of Related Art

Particles as delivery systems to transport therapeutic drugs to target sites, usually tumor or diseased tissues and cells, have been the focus of drug development for many years, as they tend to reduce toxicity and enhance bioavailability comparing to those of free drugs. Janus particles are molecules having superstructures that exhibit different chemical properties, and have been used in various applications, such as optical and electronic sensors, medicines, energy materials in batteries, and etc.

In this disclosure, the inventors take advantages in the superstructures of Janus particles by encapsulating therapeutic agents therein thereby achieving the purpose of carrying and delivering therapeutic agents to target sites, and exerting therapeutic effect at a lower dosage, as compared to that resulted by therapeutic agents that are in free form. Furthermore, reactive oxygen species (ROS) may also be generated directly from the Janus particles per se via irradiating a light thereon, thereby aids the therapeutic effect resulted from the therapeutic agents.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In general, the present disclosure relates to the discovery that a new and unique drug containing vesicle may improve therapeutic efficacy of drugs (e.g., anti-cancer drugs) encapsulated therein by prolonging their life spans, thereby achieving the same therapeutic effects at lower dosages as compared with drugs existing in free forms. Accordingly, the present disclosure provides novel drug containing vesicles, their production methods as well as methods of preventing and/or treating cancers via use of the drug containing vesicles.

The first aspect of the present disclosure aims at providing a drug-containing vesicle, which includes a carbon dot liposome (C-dot liposome) formed by a plurality of Janus particles, which are self-assembled into the C-dot liposome; and a drug encapsulated within the C-dot to liposome.

According to embodiments of the present disclosure, the plurality of Janus particles are formed by, (a) subjecting a carbon source to a heat treatment at a temperature of about 220° C. to about 250° C. until an elastomer is formed; and (b) converting the elastomer into the plurality of Janus particles by treating the elastomer with an alcohol in the presence of a base.

According to embodiments of the present disclosure, in the step (a), the carbon source is a mono-glyceride, di-glyceride or a tri-glyceride. In one preferred embodiment, the carbon source is glyceryl trioleate.

According to embodiments of the present disclosure, in the step (b), the alcohol is selected from the group consisting of methanol, ethanol, propanol, isopropanol and butanol; and the base is selected from the group consisting of sodium hydroxide, potassium hydroxide, and ammonium hydroxide.

According to embodiments of the present disclosure, the drug may be an alkylating agent, a nucleoside analogue, a topoisomerase inhibitor, a mitotic inhibitor, a proteasome inhibitor, or an interference RNA.

Examples of the alkylating agent include, but are not limited to, cyclophosphamide, chlormethine, uramustine, melphalan, chlorambucil, ifosfamide, bendamustine, carmustine, lomustine, streptozocin, ci splatin, carboplatin, dicycloplatin, eptaplatin, lobaplatin, miriplatin, and oxaliplatin.

Examples of the nucleoside analogue include, but are not limited to, didanosine, vidarabine, galidesivir, cytarabine, gemcitabine, emtricitabine, lamivudine, zalcitabine, abacavir, acyclovir, entecavir, stavudine, telbivudine, zidovudine, idoxuridine, and trifluridine.

Examples of the topoisomerase inhibitor include, but are not limited to, amsacrine, etoposide, etoposide phosphate, teniposide, doxorubicin, genistein, and ICRF-193 (i.e., 4,4′-(1,2-dimethyl-2-ethanedi)bis-2,6-pierazinedione).

Examples of the mitotic inhibitor include, but are not limited to, paclitaxel, docetaxel, vinblastine, vincristine, vindesine, vinorelbine, colchicine, podophyllotoxin, griseofulvin, and glaziovianin A.

Examples of the proteasome inhibitor include, but are not limited to, lactacystin, carfilzomib, disulfiram, epigallocatechin-3-gallate, salinosporamide A, oprozomib, delanzomib, epoxomicin, MG132, and β-hydroxy β-methylbutyrate.

The second aspect of the present disclosure aims at providing a method of producing a drug-containing vesicle. The method includes steps of, mixing a plurality of Janus particles with a drug solution to form a mixed solution; and producing the drug-containing vesicle via a film-hydration method or an injection method. In the film-hydration method, the mixed solution is condensed until a film-like structure is formed; and the film-like structure is sonicating in a salt solution to produce the drug-containing vesicle. In the injection method, the mixed solution is injected directly into a salt solution to produce the drug-containing vesicle.

According to preferred embodiments of the present disclosure, the salt solution is sodium chloride solution.

According to embodiments of the present disclosure, the drug solution is formed by dissolving a drug in a solvent, which is selected from the group consisting of water, ethanol and dimethyl sulfoxide; and the drug is an alkylating agent, a nucleoside analogue, a topoisomerase inhibitor, a mitotic inhibitor, a proteasome inhibitor, or an interference RNA.

According to optional embodiments of the present disclosure, the method further includes the step of purifying the drug-containing vesicle by dialysis, so as to remove any residual non-encapsulated drug.

According to embodiments of the present disclosure, the plurality of Janus particles are formed by, (a) subjecting a carbon source to a heat treatment at a temperature of about 220° C. to about 250° C. until an elastomer is formed; (b) converting the elastomer into the plurality of Janus particles by treating the elastomer with an alcohol in the presence of a base.

According to embodiments of the present disclosure, in the step (a), the carbon source is a mono-glyceride, di-glyceride or a tri-glyceride. In one preferred embodiment, the carbon source is glyceryl trioleate.

According to embodiments of the present disclosure, in the step (b), the alcohol is selected from the group consisting of methanol, ethanol, propanol, isopropanol and butanol; and the base is selected from the group consisting of sodium hydroxide, potassium hydroxide, and ammonium hydroxide.

According to embodiments of the present disclosure, the drug may be an alkylating agent, a nucleoside analogue, a topoisomerase inhibitor, a mitotic inhibitor, a proteasome inhibitor, or an interference RNA.

Examples of the alkylating agent include, but are not limited to, cyclophosphamide, chlormethine, uramustine, melphalan, chlorambucil, ifosfamide, bendamustine, carmustine, lomustine, streptozocin, cisplatin, carboplatin, dicycloplatin, eptaplatin, lobaplatin, miriplatin, and oxaliplatin.

Examples of the nucleoside analogue include, but are not limited to, didanosine, vidarabine, GALIDESIVIR, cytarabine, gemcitabine, emtricitabine, lamivudine, zalcitabine, abacavir, acyclovir, entecavir, stavudine, telbivudine, zidovudine, idoxuridine, and trifluridine.

Examples of the topoisomerase inhibitor include, but are not limited to, amsacrine, etoposide, etoposide phosphate, teniposide, doxorubicin, genistein, and ICRF-193.

Examples of the mitotic inhibitor include, but are not limited to, paclitaxel, docetaxel, vinblastine, vincristine, vindesine, vinorelbine, colchicine, podophyllotoxin, griseofulvin, and glaziovianin A.

Examples of the proteasome inhibitor include, but are not limited to, lactacystin, carfilzomib, disulfiram, epigallocatechin-3-gallate, salinosporamide A, oprozomib, delanzomib, epoxomicin, MG132, and β-hydroxy β-methylbutyrate.

The third aspect of the present disclosure aims at providing a method for treating a subject afflicted with a cancer.

According to some embodiments, the method comprises the step of, administering to the subject an effective amount of the drug containing vesicle of the present disclosure to suppress the growth of the cancer. Optionally or additionally, the method further includes a step of irradiating the subject in sequence with a first and a second light respectively having a wavelength of 350-400 nm and 480-550 nm. Preferably, the first light has the wavelength of 385 nm, while the second light has the wavelength of 530 nm.

According to other embodiments, the method comprises administering to the subject an effective amount of a carbon dot liposome (C-dot liposome) formed by a plurality of Janus particles, which are self-assembled into the C-dot liposome, and irradiating the subject in sequence with a first and a second light respectively having a wavelength of 350-400 nm and 480-550 nm. The plurality of Janus particles are formed by, (a) subjecting a carbon source to a heat treatment at a temperature of about 220° C. to about 250° C. until an elastomer is formed; (b) converting the elastomer into the plurality of Janus particles by treating the elastomer with an alcohol in the presence of a base.

Examples of the cancer suitable for treating by the present method include, but are not limited to, analplastic large cell lymphoma, angiosarcoma, bone cancer, bladder cancer, biliary cancer, brain cancer, breast cancer, cancer of testicles, cancer of connective tissue, cancer of retina, colon cancer, cervical cancer, endometrial cancer, epidermal carcinoma, esophageal squamous cell carcinoma, follicular dentritic cell carcinoma, fallopian tube cancer, gastrointestinal stromal tumor (GIST), glioma, glioblastoma, head and neck cancer, hematopoietic tumors of lymphoid lineage, heptatocellular carcinoma, intestinal cancer, Kaposi's sarcoma, keratoacanthomas, Li-Fraumeni syndrome, lung cancer, malignant ascites, melanoma, mesothelioma, myeloid leukemia, myelodysplastic syndrome (MDS), myelodysplasia, muscle invasive cancer, nasopharyngeal, neuroendocrine cancer, neuroblastoma, oesophagogastric, ovary cancer, pancreatic cancer, peritoneal cancer, papillary serous mullerian cancer, prostate cancer, prostatic hypertrophy, renal cancer, seminal vesicle tumor, spleen cancer, stomach cancer, small bowel cancer, salivary gland cancer, thyroid cancer, teratcarcinoma, thyroid follicular cancer, tumor of mesenchymal origin, uveal melanoma, uterine sarcoma, Von Hippel-Lindau syndrome (VHL), and Waldenstrom' s macroglobulinemia.

Examples of the hematopoietic tumors of lymphoid lineage include, but are not limited to, leukemia, acute lymphocytic leukemia, chronic lymphocytic leukemia, B-cell lymphoma, Burkitt's lymphoma, multiple myeloma, Hodgkin's lymphoma, and Non-Hodgkin's lymphoma.

Examples of the myeloid leukemia include, but are not limited to, acute myelogenous leukemia (AML) and chronic myelogenous leukemia (CIVIL).

According to embodiments of the present disclosure, the subject is a mammal, preferably a human.

Many of the attendant features and advantages of the present disclosure will become better understood with reference to the following detailed description considered in connection with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present description will be better understood from the following detailed description read in light of the accompanying drawings, where:

FIG. 1A is a bar graph depicting the intracellular oxidative stress in prostate cancer cells treated the C-dot liposomes of Example 1.1 in accordance with one embodiment of the present disclosure;

FIG. 1B are images of prostate cancer cells of FIG. 1A taken before and after exposure to UV light (385 nm) and a light of 530 nm in accordance with one embodiment of the present disclosure; and

FIG. 2 are bar graphs depicting cell viability of prostate cancer cells (A) and normal fibroblast cells (B) treated with the C-dot liposomes of Example 1.1 in accordance with one embodiment of the present disclosure.

In accordance with common practice, the various described features/elements are not drawn to scale but instead are drawn to best illustrate specific features/elements relevant to the present invention. Also, like reference numerals and designations in the various drawings are used to indicate like elements/parts.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

1. Definitions

For convenience, certain terms employed in the context of the present disclosure are collected here. Unless defined otherwise, all technical and scientific terms used herein have the same meaning as commonly understood by one of the ordinary skill in the art to which this invention belongs.

The term “mono-glyceride”, “di-glyceride” or “tri-glyceride” as used herein is meant the glycerol mono-ester, di-esters or tri-esters of and the same or mixed fatty acids. Fatty acid refers to straight chain saturated or unsaturated monocarboxylic acids having a carbon chain length of from C₁₂ to C₃₀, such as Laurie acid, myristic acid, myristoleic aicd, palmitic acid, palmitoleic aicd, stearic acid, oleic acid, linoleic acid, arachidic acid, arachidonic acid, and etc.

The term “treatment” as used herein are intended to mean obtaining a desired pharmacological and/or physiologic effect, e.g., delaying or inhibiting the metastasis of a cancer. The effect may be prophylactic in terms of completely or partially preventing a disease or symptom thereof and/or therapeutic in terms of a partial or complete cure for a disease and/or adverse effect attributable to the disease. “Treatment” as used herein includes preventative (e.g., prophylactic), curative or palliative treatment of a disease in a mammal, particularly human; and includes: (1) preventative (e.g., prophylactic), curative or palliative treatment of a disease or condition (e.g., a cancer or heart failure) from occurring in an individual who may be pre-disposed to the disease but has not yet been diagnosed as having it; (2) inhibiting a disease (e.g., by arresting its development); or (3) relieving a disease (e.g., reducing symptoms associated with the disease).

The term “administered”, “administering” or “administration” are used interchangeably herein to refer a mode of delivery, including, without limitation, intraveneously, intramuscularly, intraperitoneally, intraarterially, intracranially, or subcutaneously administering an agent (e.g., the drug containing vesicles of the present disclosure) that suppresses the growth of a cancer.

The term “an effective amount” as used herein refers to an amount effective, at dosages, and for periods of time necessary, to achieve the desired result with respect to the treatment of a cancer. For example, in the treatment of a cancer, an agent (i.e., the present drug-containing vesicle) is administered in an amount that effectively decrease, prevents, delays or suppresses or arrests the growth of the cancerous cells. An effective amount of an agent is not required to cure a disease or condition but will provide a treatment for a disease or condition such that the onset of the disease or condition is delayed, hindered or prevented, or the disease or condition symptoms are ameliorated. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the like. Effective amount may be expressed, for example, as the total mass of the active agent (e.g., in grams, milligrams or micrograms) or a ratio of mass of the active agent to body mass, e.g., as milligrams per kilogram (mg/kg). The effective amount may be divided into one, two or more doses in a suitable form to be administered at one, two or more times throughout a designated time period.

The term “subject” or “patient” is used interchangeably herein and is intended to mean a mammal including the human species that is treatable by the compound of the present invention.

The term “mammal” refers to all members of the class Mammalia, including humans, primates, domestic and farm animals, such as rabbit, pig, sheep, and cattle; as well as zoo, sports or pet animals; and rodents, such as mouse and rat. Further, the term “subject” or “patient” intended to refer to both the male and female gender unless one gender is specifically indicated. Accordingly, the term “subject” or “patient” comprises any mammal which may benefit from the treatment method of the present disclosure. Examples of a “subject” or “patient” include, but are not limited to, a human, rat, mouse, guinea pig, monkey, pig, goat, cow, horse, dog, cat, bird and fowl. In a preferred embodiment, the subject is a human.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The singular forms “a,” “and,” and “the” are used herein to include plural referents unless the context clearly dictates otherwise.

2. Detail Description of Preferred Embodiments

The present disclosure is based, at least in part, on the discovery that a new and unique drug containing vesicle may improve therapeutic efficacy of drugs (e.g., anti-cancer drugs) encapsulated therein by preventing them from degradation thereby achieving the same therapeutic effects at lower dosages as compared with drugs existing in free forms. Accordingly, the present disclosure provides novel drug containing vesicles, their production methods as well as methods of preventing and/or treating cancers via use of the drug containing vesicles.

2.1 Carbon Dot Liposomes and their Preparation Methods

(i) Carbon Dot Liposomes

The present disclosure aims at providing a carbon dot liposome formed by a plurality of Janus particles, which are capable of self-assembling into the C-dot liposome; as well as a drug-containing vesicle, which includes the carbon dot liposome (C-dot liposome); and a drug encapsulated within the C-dot liposome.

Janus particles are asymmetric particles of sub-micron or micron-sized parts having two chemical properties and/or different polarities. Because of these properties, Janus particles are a unique class of materials useful in many applications ranging from catalysis to therapeutic treatments. The present disclosure takes advantages in the unique properties of Janus particles, which are self-assembled into liposomes, allowing them to act as drug delivery systems.

According to embodiments of the present disclosure, the plurality of Janus particles are formed by, (a) subjecting a carbon source to a heat treatment at a temperature of about 220° C. to about 250° C. until an elastomer is formed; and (b) converting the elastomer into the plurality of Janus particles by treating the elastomer with an alcohol in the presence of a base.

According to embodiments of the present disclosure, in the step (a), the carbon source is any of a mono-glyceride, di-glyceride or tri-glyceride. Preferably, the carbon source is a tri-glyceride formed by glycerol and three straight chain saturated or unsaturated onocarboxylic acids, each having a carbon chain length of from C₁₂ to C₃₀. In one preferred embodiment, glyceryl trioleate was heated at a temperature of about 220° C. for 3 days until an elastomer is formed.

According to embodiments of the present disclosure, in the step (b), the thus formed elastomer in the step (a) is converted into Janus particles by treating with an alcohol solution in the presence of a base. Examples of the alcohol include, but are not limited to, methanol, ethanol, propanol, isopropanol and butanol. Examples of the base include, but are not limited to, sodium hydroxide, potassium hydroxide, and ammonium hydroxide. In one preferred embodiment, the elastomer in the step (a) is converted into Janus particles by subjecting to the treatment of an ethanol solution in the presence of NaOH. The thus produced Janus particles will automatically assemble into liposomes (i.e., C-dot liposomes).

(ii) Drug-Containing Vesicles

The automatically assembled C-dot liposomes described above may serve as vesicles for carrying and/or delivering therapeutic drugs. To this purpose, therapeutic drugs (e.g., anti-cancer drugs) are encapsulated in the C-dot liposomes via a film-hydration method or an injection method.

In the film-hydration method, the present C-dot liposomes, which are produced by a plurality of self-assembled Janus particles described above, is first mixed with a drug solution to form a mixed solution, which is condensed to reduce the volume until a film-like structure is produced. The film-like structure is then sonicated in a salt solution (e.g., 0.9% NaCl) to produce the desired drug-containing vesicles.

In the injection method, a mixed solution of the C-dot liposomes and the drug is produced by the same procedures described above in the film-hydration method. However, unlike the condensation steps required by the film-hydration method, the mixed solution in this embodiment is rapidly injected into a salt solution (e.g., 0.9% NaCl) to produce the desired drug-containing vesicles.

According to embodiments of the present disclosure, the drug solution is formed by dissolving a drug, such as an alkylating agent, a nucleoside analogue, a topoisomerase inhibitor, a mitotic inhibitor, a proteasome inhibitor, and an interference RNA, in a solvent. Depending on the solubility of the drug in a particular solvent, examples of solvent suitable for use in the present disclosure include, but are not limited to, water, ethanol and dimethyl sulfoxide (DMSO).

According to embodiments of the present disclosure, the drug may be an alkylating agent, a nucleoside analogue, a topoisomerase inhibitor, a mitotic inhibitor, a proteasome inhibitor, or an interference RNA. Examples of the alkylating agent include, but are not limited to, cyclophosphamide, chlormethine, uramustine, melphalan, chlorambucil, ifosfamide, bendamustine, carmustine, lomustine, streptozocin, cisplatin, carboplatin, dicycloplatin, eptaplatin, lobaplatin, miriplatin, and oxaliplatin. Examples of the nucleoside analogue include, but are not limited to, didanosine, vidarabine, galidesivir, cytarabine, gemcitabine, emtricitabine, lamivudine, zalcitabine, abacavir, acyclovir, entecavir, stavudine, telbivudine, zidovudine, idoxuridine, and trifluridine. Examples of the topoisomerase inhibitor include, but are not limited to, amsacrine, etoposide, etoposide phosphate, teniposide, doxorubicin, genistein, and ICRF-193. Examples of the mitotic inhibitor include, but are not limited to, paclitaxel, docetaxel, vinblastine, vincristine, vindesine, vinorelbine, colchicine, podophyllotoxin, griseofulvin, and glaziovianin A. Examples of the proteasome inhibitor include, but are not limited to, lactacystin, carfilzomib, disulfiram, epigallocatechin-3-gallate, salinosporamide A, oprozomib, delanzomib, epoxomicin, MG132, and β-hydroxy β-methylbutyrate.

According to optional embodiments of the present disclosure, the drug containing vesicles, which may be produced by the film-hydration method or the injection method, may be further purified by dialysis, so as to remove any residual non-encapsulated drugs.

2.2 Methods for Treating Cancers

The present disclosure also aims at providing a therapeutic treatment to a subject afflicted with a cancer. To this purpose, the present C-dot liposome or the drug containing vesicles (i.e., C-dot liposomes encapsulated therein anti-cancer agents) are used as medicaments for the treatment of cancers. The present disclosure thus encompasses a method for treating a subject afflicted with a cancer.

(i) Treating Cancers via C-Dot Liposomes

In some embodiments, the method comprises (a) administering to the subject, an effective amount of the C-dot liposomes, and (b) irradiating the subject in sequence with a firs and a second light respectively having a wavelength of 350-400 nm and 480-550 nm. In such embodiments, reactive oxygen species (ROS) are generated directly from the C-dot liposomes, thereby achieving the effect of suppressing cancer growth.

Preferably, the first light has the wavelength between 350 to 400 nm, such as 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399 and 400 nm; more preferably, between 360 to 390 nm, such as 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, and 390 nm; most preferably, the first light has the wavelength of 385 nm.

Preferably, the second light has the wavelength between 480 to 550 nm, such as 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, and 550 nm; more preferably, between 490 to 540 nm, such as 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, and 540 nm; most preferably, the second light has the wavelength of 530 nm.

According to embodiments of the present disclosure, the subject may be irradiated in sequence with the first and second light independently for 10 seconds to 15 minutes, such as 10, 20, 30, 40, 50, and 60 seconds, 1, 2, 3, 4, 5, 6, 7, 8, 9, 10, 11, 12, 13, 14 and 15 minutes; preferably for 30 seconds to 12 minutes, such as 30, 40, 50, and 60 seconds, 1, 2, 3, 4, 5, 6, 7, 8, 9, 10, 11, and 12 minutes. According to one preferred embodiment, the subject is irradiated with the first light of 385 nm for 5 minutes, then with the second light of 530 nm for 10 minutes. According to another preferred embodiment, the subject is irradiated with the first light of 385 nm for 1 minute, then with the second light of 530 nm for 5 minutes.

Examples of the cancer suitable for treating by the present method include, but are no tlimited to, analplastic large cell lymphoma, angiosarcoma, bone cancer, bladder cancer, biliary cancer, brain cancer, breast cancer, cancer of testicles, cancer of connective tissue, cancer of retina, colon cancer, cervical cancer, endometrial cancer, epidermal carcinoma, esophageal squamous cell carcinoma, follicular dentritic cell carcinoma, fallopian tube cancer, gastrointestinal stromal tumor (GIST), glioma, glioblastoma, head and neck cancer, hematopoietic tumors of lymphoid lineage, heptatocellular carcinoma, intestinal cancer, Kaposi's sarcoma, keratoacanthomas, Li-Fraumeni syndrome, lung cancer, malignant ascites, melanoma, mesothelioma, myeloid leukemia, myelodysplastic syndrome (MDS), myelodysplasia, muscle invasive cancer, nasopharyngeal, neuroendocrine cancer, neuroblastoma, oesophagogastric, ovary cancer, pancreatic cancer, peritoneal cancer, papillary serous mullerian cancer, prostate cancer, prostatic hypertrophy, renal cancer, seminal vesicle tumor, spleen cancer, stomach cancer, small bowel cancer, salivary gland cancer, thyroid cancer, teratcarcinoma, thyroid follicular cancer, tumor of mesenchymal origin, uveal melanoma, uterine sarcoma, Von Hippel-Lindau syndrome (VHL), and Waldenstrom' s macroglobulinemia.

Examples of the hematopoietic tumors of lymphoid lineage include, but are not limited to, leukemia, acute lymphocytic leukemia, chronic lymphocytic leukemia, B-cell lymphoma, Burkitt's lymphoma, multiple myeloma, Hodgkin's lymphoma, and Non-Hodgkin's lymphoma.

Examples of the myeloid leukemia include, but are not limited to, acute myelogenous leukemia (AML) and chronic myelogenous leukemia (CIVIL).

According to embodiments of the present disclosure, the subject is or has been afflicted with cancer, which may be any of breast cancer, cervical cancer, colon cancer, lung cancer, hepatic cancer, and pancreatic cancer.

(ii) Treating Cancers via Drug-Containing Vesicles

In some embodiments of the present disclosure, the method comprises, administering to the subject an effective amount of the present drug containing vesicles (e.g., C-dot liposomes encapsulated therein an anti-cancer drug), so that the growth of the cancer is suppressed.

Additionally or optionally, the method further includes a step of irradiating the subject in sequence with a firs and a second light respectively having a wavelength of 350-400 nm and 480-550 nm, so that the growth of the cancer is suppressed.

Preferably, the first light has the wavelength between 350 to 400 nm, such as 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399 and 400 nm; more preferably, between 360 to 390 nm, such as 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, and 390 nm; most preferably, the first light has the wavelength of 385 nm.

Preferably, the second light has the wavelength between 480 to 550 nm, such as 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, and 550 nm; more preferably, between 490 to 540 nm, such as 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, and 540 nm; most preferably, the second light has the wavelength of 530 nm.

According to embodiments of the present disclosure, the subject may be irradiated in sequence with the first and second light independently for 10 seconds to 15 minutes, such as 10, 20, 30, 40, 50, and 60 seconds, 1, 2, 3, 4, 5, 6, 7, 8, 9, 10, 11, 12, 13, 14 and 15 minutes; preferably for 30 seconds to 12 minutes, such as 30, 40, 50, and 60 seconds, 1, 2, 3, 4, 5, 6, 7, 8, 9, 10, 11, and 12 minutes. According to one preferred embodiment, the subject is irradiated with the first light of 385 nm for 5 minutes, then with the second light of 530 nm for 10 minutes. According to another preferred embodiment, the subject is irradiated with the first light of 385 nm for 1 minute, then with the second light of 530 nm for 5 minutes.

Examples of the cancer suitable for treating by the present method include, but are no tlimited to, analplastic large cell lymphoma, angiosarcoma, bone cancer, bladder cancer, biliary cancer, brain cancer, breast cancer, cancer of testicles, cancer of connective tissue, cancer of retina, colon cancer, cervical cancer, endometrial cancer, epidermal carcinoma, esophageal squamous cell carcinoma, follicular dentritic cell carcinoma, fallopian tube cancer, gastrointestinal stromal tumor (GIST), glioma, glioblastoma, head and neck cancer, hematopoietic tumors of lymphoid lineage, heptatocellular carcinoma, intestinal cancer, Kaposi's sarcoma, keratoacanthomas, Li-Fraumeni syndrome, lung cancer, malignant ascites, melanoma, mesothelioma, myeloid leukemia, myelodysplastic syndrome (MDS), myelodysplasia, muscle invasive cancer, nasopharyngeal, neuroendocrine cancer, neuroblastoma, oesophagogastric, ovary cancer, pancreatic cancer, peritoneal cancer, papillary serous mullerian cancer, prostate cancer, prostatic hypertrophy, renal cancer, seminal vesicle tumor, spleen cancer, stomach cancer, small bowel cancer, salivary gland cancer, thyroid cancer, teratcarcinoma, thyroid follicular cancer, tumor of mesenchymal origin, uveal melanoma, uterine sarcoma, Von Hippel-Lindau syndrome (VHL), and Waldenstrom's macroglobulinemia.

Examples of the hematopoietic tumors of lymphoid lineage include, but are not limited to, leukemia, acute lymphocytic leukemia, chronic lymphocytic leukemia, B-cell lymphoma, Burkitt's lymphoma, multiple myeloma, Hodgkin's lymphoma, and Non-Hodgkin's lymphoma.

Examples of the myeloid leukemia include, but are not limited to, acute myelogenous leukemia (AML) and chronic myelogenous leukemia (CIVIL).

According to embodiments of the present disclosure, the subject is or has been afflicted with cancer, which may be any of breast cancer, cervical cancer, colon cancer, lung cancer, hepatic cancer, and pancreatic cancer.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent.

### EXAMPLES

### Materials and Methods

Cell lines and cell culture. A549, Hela, MCF-7, Huh7, C2BBe1, BxPC-3, A375.S2, CCRF-CEM, TrampC1 and NIH 3T3 cells were obtained from the American Type Culture Collection (ATCC; Manassas, Va., USA). A549, Hela, Huh7, A375.S2, TrampC1, and NIH 3T3 cells were maintained in Dulbecco's minimal essential medium (DMEM) supplemented with 1.5 g/L sodium bicarbonate,10% fetal bovine serum (FBS), 1.0% antibiotic-antimycotic, L-glutamine (2.0×10⁻³ M), and 1.0% nonessential amino acids. MCF-7, BxPC-3 and CCRF-CEM cells were cultured in Roswell Park Memorial Institute (RPMI) medium supplemented with 10% FBS, and 1.0% antibiotic-antimycotic. C2BBe1 cells were grown in DMEM 1.5 g/L sodium bicarbonate, 1.0% antibiotic-antimycotic, L-glutamine (2.0×10⁻³ M), and 1.0% nonessential amino acids, 10 mg/L human holo-transferrin, and 10% fetal bovine serum. The cell number and viability of the cells were determined by the trypan blue exclusion method and Alamar Blue assay, respectively.

Alamar Blue Assay.

The assay is designed to measure quantitatively the proliferation of cell by use of a fluorometric/colorimetric growth indicator based on detection of metabolic activity. Specifically, the alamar blue system incorporates an oxidation-reduction (REDOX) indicator that both fluoresces and changes color in response to chemical reduction of growth medium resulting from cell growth. As cells being tested grow, innate metabolic activity results in a chemical reduction of the system. Continued growth maintains a reduced environment while inhibition of growth maintains an oxidized environment. Reduction related to growth causes the REDOX indicator to change from oxidized (non-fluorescent, blue) form to reduced (fluorescent, red) form.

Briefly, cells (2,000 cess/well) were grown to confluency in their growth media, then the present C-dot liposomes or drug-containing vesicles were added and incubated for about 10 minutes, then cells were irradiated with a light of 385 nm for 1 min, and subsequently with a light of 530 nm for 5 minutes. The medium was then removed and replaced with a fresh one. Fresh media was also added to a sterile flask containing no cells to serve as a negative control. All flasks were then re-incubated at 37° C., 5% CO₂ for additional 24 hrs. At the end of the incubation, alamarBlue® agents was added to each flask and reacted for 2 hrs. Fluorescence of the as-formed reduced dye was measured using a Synergy H1 microplate spectrophotometer, with an excitation wavelength of 560 nm and an emission wavelength of 590 nm. As the fluorescence intensity is directly correlated with cell quantity, thus cell viability is calculated by assuming 100% viability in the control cells (in which the culture media was devoid of any anti-cancer drug).

### Example 1

### Preparation of Drug Containing Vesicles

1.1 Preparation of Carbon Dot Liposomes

C-dot liposomes were prepared in accordance with the procedures described in US 2017/0354612A1. Briefly, glyceryl trioleate (50 g) was heated at 220° C. for 3 days until it turned into carbon dots, which were subsequently converted into Janus particles by the treatment of 0.2M NaOH/ethanol solution. The Janus particles were then subjected to the treatment of a 0.9% NaCl solution to form the C-dot liposomes.

1.2 Encapsulating an Anti-Cancer Drug into the C-Dot Liposomes of Example 1.1 via Film-Hydration Method

A drug solution was prepared by dissolving an anti-cancer drug (e.g., cisplatin) in a solvent (e.g., water, ethanol, and dimethyl sulfoxide (DMSO) at a concentration of 0.5 mg drug/mL solvent; then the drug solution was mixed with the C-dot liposomes of example 1.1 in a ratio of about 1:80, which was concentrated until a film-like structure was formed. Then, a 0.9% NaCl solution (1.6 mL) was added to the film-like structure and the mixture was sonicated to form the desired drug containing vesicles.

1.3 Encapsulating an Anti-Cancer Drug into the C-Dot Liposomes of Example 1.1 via Injection Method

The C-dot liposomes of example 1.1 was mixed with the drug solution prepared in accordance with steps described in example 1.2 in a ratio of 1 to 10, the mixture was then injected into 0.9% NaCl solution (2 mL) with stirring until the desired drug containing vesicles were formed.

1.4 Characterization of the Drug Containing Vesicles of Examples 1.2 and 1.3

1.4.1 Encapsulating Ratio and Drug Load

The drug containing vesicles respectively produced in examples 1.2 and 1.3 were subjected to analysis to determine the respective drug load and the encapsulating ratio. To these purpose, the drug containing vesicles were dialyzed against 0.9% NaCl, which was in excess about 100 folds in volume than that of the drug containing vesicles, to remove non-encapsulated drug molecules, then the drug molecules loaded therein were released by the treatment of 50% ethanol, and amount of drugs released therefrom were then determined by high performance liquid chromatography (HPLC), except cisplatin, which was quantified by inductively coupled plasma mass spectrometry (ICP MS). Results are summarized in Table 1.

TABLE 1
The respective encapsulating ratio and drug load of
the drug containing vesicles of examples 1.2 and 1.3

| | | Solubility of | Encapsulating | Drug Load |
|---|---|---|---|---|
| Anti-cancer | Preparation | the anti-cancer | Ratio | (DL) |
| drug | method | drug (mg/L) | (ER) (%)¹ | (%)² |
| Cisplatin | Example 1.2 | 2,500 | 81.9 | 1.0 |
| Gemcitabine | Example 1.2 | 51,000 | 64.9 | 6.1 |
| Doxorubicin | Example 1.2 | 2,600 | 82.5 | 8.5 |
| Docetaxel | Example 1.3 | 0.27 | 36.2 | 3.5 |
| Paclitaxel | Example 1.3 | 5.6 | 33.2 | 3.2 |
| Carfilzomib | Example 1.3 | <0.0012 | 75.9 | 6.8 |
| Anti-survivin | Example 1.2 | — | 95.1 | — |
| siRNA | | | | |

¹ER (%) = the amount of a drug encapsulated in the C-dot liposome/the total amount of the drug
²DL (%) = the weight of a drug encapsulated in the C-dot liposome/the total weight of the drug and the C-dot liposome

It is evident from the data presented in Table 1, the drug encapsulating ratio in the drug containing vesicles produced by the film-hydration method of example 1.2 or by the injection method of example 1.3 ranged from about 25.9% to about 95.1%, with a drug load that ranged from about 1% to about 3.5%.

1.4.2 Degradation of Drugs

In this example, the degradation of drug molecules encapsulated in the drug containing vesicles and that of free drugs were investigated. To this purpose, the drug containing vesicles of examples 1.2 or 1.3 or the free drug were placed in water, and let standing for 48 hrs, then the amounts of each drugs in the water were measured by HPLC or ICP MS. Results are summarized in Table 2.

TABLE 2
Percentage of drugs remained un-degraded

| | | Drug containing vesicles of |
|---|---|---|
| | Free drug (%) | examples 1.2 or 1.3 (%) |
| Doxorubicin | 69.5 | 100 |
| Gemcitabine | 58.8 | 99.3 |
| Docetaxel | 83.6 | 100 |
| Carfilzomib | 48.5 | 90.2 |

It is evident that the present C-dot liposomes may protect the drugs encapsulated therein from degradation, in which over 90% of drugs remained un-degraded, while about 17% to 55% of free drugs were degraded.

### Example 2

### The Drug Containing Vesicles of Examples 1.2 or 1.3 Suppress the Growth of Cancer Cells

In this example, the anti-cancer activity of the drug containing vesicles of Examples 1.2 or 1.3 was investigated. To this purpose, cancer cell lines, including A549, Hela, MCF-7, Huh7, C2BBe1, BxPC-3, A375.S2, and CCRF-CEM cells, were respectively plated at the concentration of about 10⁴ cells/well in a culture medium for 24 h at 37° C. in an atmosphere containing 5% CO₂, the culture media was then replaced by a culture medium containing anti-cancer drugs or the drug containing vesicles of example 1, and further cultured for additional 48 hr. Cell viability was determined by Alamar Blue assay in accordance with the manufacturer's protocols. Results are summarized in Table 3.

TABLE 3
Efficacy of the drug containing vesicles of Examples
1.2 or 1.3 on the growth of cancerous cells

| | | | Anti-cancer Drug | | | | |
|---|---|---|---|---|---|---|---|
| | | | Cisplatin | Gemcitabine | Doxorubicin | Docetaxel | Carfilzomib |
| IC₅₀ | A375 | Examples | 6.3 | 6.3 | 0.08 | 7.4 | 78 |
| (μM) | | 1.2 or 1.3 | | | | | |
| | | Free drug | 9.2 | 66 | 0.75 | 29 | 750 |
| | C2BBE1 | Examples | 7.7 | 66 | 1.1 | 13 | 0.96 |
| | | 1.2 or 1.3 | | | | | |
| | | Free drug | 11 | 170 | 13 | 100 | 1.6 |
| | A549 | Examples | 4.7 | 6.0 | 0.01 | 0.1 | 0.33 |
| | | 1.2 or 1.3 | | | | | |
| | | Free drug | 6.5 | 17 | 0.04 | 1 | 1.8 |
| | HeLa | Examples | 4.0 | 7.2 | 0.5 | 0.1 | 0.57 |
| | | 1.2 or 1.3 | | | | | |
| | | Free drug | 3.1 | 94 | 1.7 | 0.67 | 1.3 |
| | Huh7 | Examples | 4.2 | 1.3 | 0.17 | 0.01 | 0.1 |
| | | 1.2 or 1.3 | | | | | |
| | | Free drug | 6.3 | 1 | 0.49 | 0.01 | 0.7 |
| | BxPC3 | Examples | 2.5 | 17 | 0.11 | 0.49 | 0.01 |
| | | 1.2 or 1.3 | | | | | |
| | | Free drug | 4.8 | 51 | 0.58 | 4.7 | 0.08 |
| | MCF7 | Examples | 4.4 | 16 | 0.31 | 1.5 | 0.01 |
| | | 1.2 or 1.3 | | | | | |
| | | Free drug | 33 | 48 | 2.0 | 6.3 | 0.1 |

It was found that the drug containing vesicles of Example 1 improved the efficacies of anti-cancer drugs in suppressing the growth of various types of cancerous cells, including the lung cancer cells A549, cervical cancer cells Hela, breast cancer cells MCF7, hepatoma cells Huh7, colon cancer cells C2BBE1, and pancreatic cancer cells BxPC3, by lowering the ICso values of each anti-cancer drugs (encapsulated in C-dot liposomes vs free forms).

### Example 3

### The C-Dot Liposome of Example 1.1 Suppresses the Growth of Cancer Cells

3.1 Generation of Reactive Oxygen Species (ROS)

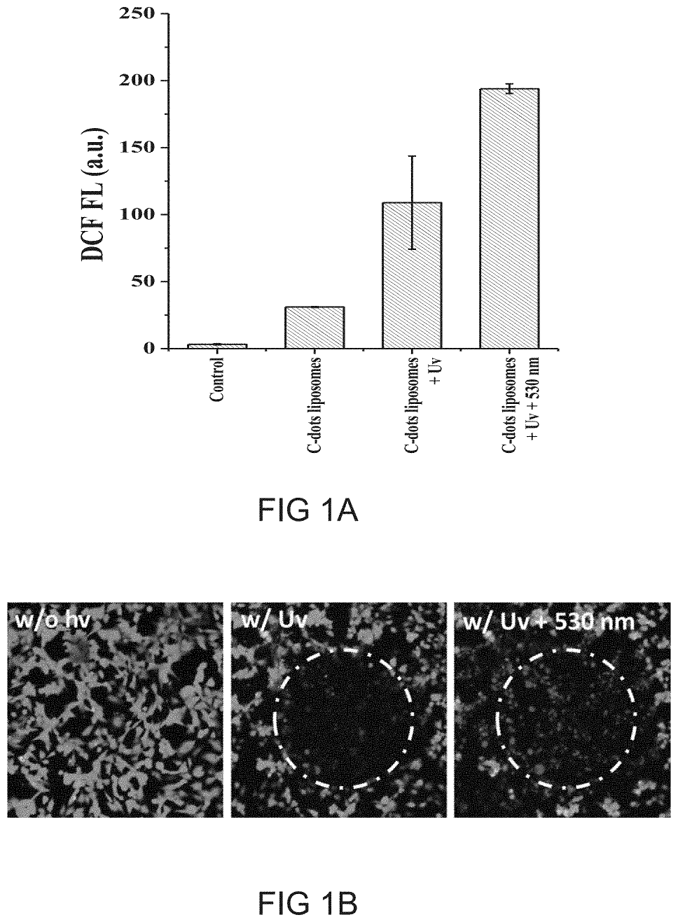

In this example, the anti-cancer activity of the C-dot liposomes of Example 1.1 was investigated. To this purpose, cancer cell lines, including TrampC1 and NIH 3T3 cells, were respectively plated at the concentration of about 4×10⁴ cells/well in a culture medium for 12 hrs at 37° C. in an atmosphere containing 5% CO2, the culture media was then replaced by a culture medium containing DCFH-DA probes (which are indicators for intracellular oxidative stress) (25 μM) and cultivated for 30 minutes, the dyes were then removed and cells were rinsed thoroughly with PBS, then C-dot liposomes of example 1.1 were added and reacted for 10 minutes. The cells were then irradiated with a UV light of 385 nm for 1 minutes, and then with a light of 530 nm for 5 minutes. The cells were let stand for additional 30 minutes, before the C-dot liposomes were removed by washing with PBS. Cells were then harvested by enzyme (i.e., trypsin-EDTA) treatment, and analyzed via flow cytometry. Quantitated results are illustrated in FIG. 1.

It was found that treatment of C-dot liposomes of Example 1.1 per se caused an increase in intracellular oxidative stress (i.e., generation of intracellular H₂O₂ or reactive oxygen species (ROS)) in TramPC1 cells, which was manifested by an increase in the intensity of DCFH-DA probes, the stress further increased for about 3-folds as cells were exposed to light at 385 nm, and to about 8-folds with subsequent exposure to light at 530 nm (See FIG. 1A). It was thus postulated that the thus generated ROS could be applied to killing cancer cells.

To confirm the cell-killing effect of ROS generated from the C-dot liposomes of example 1.1, TrampC1 cells (10⁵ cells/well) were cultivated for 24 hrs, and then treated with Calcein AM/PI agents (which stained both the live and dead cells) and C-dot liposomes (400 μg/mL). The cells were returned to culture for additional 30 minutes, then irradiated with a UV light of 385 nm for 5 minutes, let stand for 1 hr, then irradiated with a light of 530 nm for 10 minutes, let stand for another hr, before taking the images.

As revealed by the images in FIG. 1B, cell viability in the area where they were exposed to light (385 nm and subsequently at 530 nm) is extremely low, as compared with that in the area not exposed to light. The results confirmed that the C-dot liposomes of example 1.1 per se possessed anti-cancer cell activity as they caused oxidative stress in cells, the stress may be further increased by exposing the C-dot liposomes of example 1.1 to light independently at 385 nm and 530 nm, which led to the generation of ROS, that wiped out the cells in the light exposed area.

3.2 Toxicity Evaluation

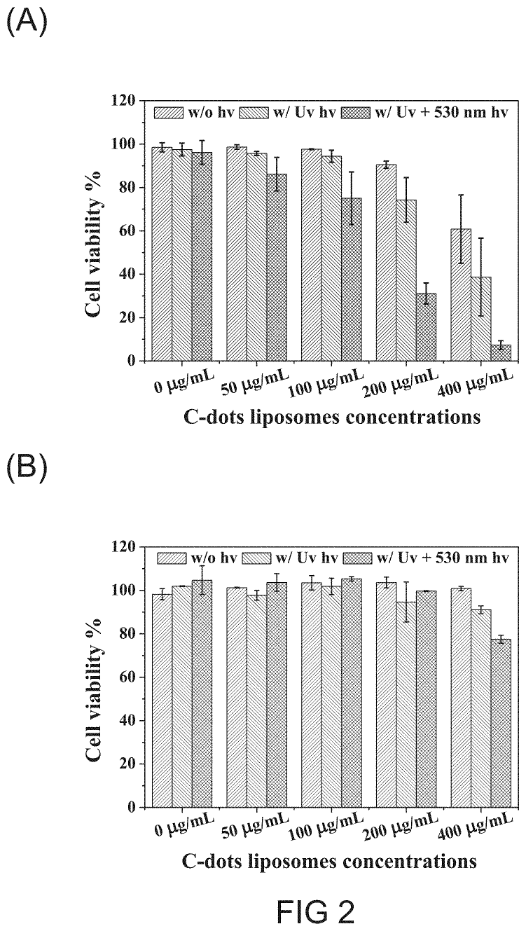

As the data in Example 3.1 indicated, the C-dot liposomes of example 1.1 generated ROS upon being exposed to light, thus the cytotoxicity effect of the C-dot liposomes of example 1.1 on TrampC1 cancer cells and normal NIH 3T3 cells was independently determine by use of Alamar Blue assay. Results are illustrated in FIG. 2, in which FIG. 2A are results from cancer cells, while FIG. 2B are results from normal cells.

Surprisingly, it was found that the C-dot liposomes of example 1.1 were toxic only to cancer cells, as cell viability of TrampC1 cells decreased with an increase in the concertation of the C-dot liposomes and exposure to light (FIG. 2A); by contrast, cell viability of normal 3T3 cells remained relatively the same regardless of an increase in the concentration of the C-dot liposomes or exposure of light (DIG 2B).

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A drug-containing vesicle comprising:
a carbon dot liposome (C-dot liposome) formed by a plurality of Janus particles, which are self-assembled into the C-dot liposome; and
a drug encapsulated within the C-dot liposome.

2. The drug-containing vesicle of claim 1, wherein the plurality of Janus particles are formed by,
(a) subjecting a carbon source to a heat treatment at a temperature of about 220° C. to about 250° C. until an elastomer is formed;
(b) converting the elastomer into the plurality of Janus particles by treating the elastomer with an alcohol in the presence of a base.

3. The drug-containing vesicle of claim 2, wherein in the step (a), the carbon source is a mono-glyceride, di-glyceride or a tri-glyceride.

4. The drug-containing vesicle of claim 3, wherein the carbon source is glyceryl trioleate.

5. The drug-containing vesicle of claim 2, wherein in the step (b), the alcohol is selected from the group consisting of methanol, ethanol, propanol, isopropanol and butanol; and
the base is selected from the group consisting of sodium hydroxide, potassium hydroxide, and 20 ammonium hydroxide.

6. The drug-containing vesicle of claim 1, wherein the drug is an alkylating agent, a nucleoside analogue, a topoisomerase inhibitor, a mitotic inhibitor, a proteasome inhibitor, or an interference RNA.

7. The drug-containing vesicle of claim 6, wherein the alkylating agent is cyclophosphamide, chlormethine, uramustine, melphalan, chlorambucil, ifosfamide, bendamustine, carmustine, lomustine, streptozocin, cisplatin, carboplatin, dicycloplatin, eptaplatin, lobaplatin, miriplatin, or oxaliplatin;
the nucleoside analogue is didanosine, vidarabine, galidesivir, cytarabine, gemcitabine, emtricitabine, lamivudine, zalcitabine, abacavir, acyclovir, entecavir, stavudine, telbivudine, zidovudine, idoxuridine, or trifluridine;
the topoisomerase inhibitor is amsacrine, etoposide, etoposide phosphate, teniposide, doxorubicin, genistein, or ICRF-193;
the mitotic inhibitor is paclitaxel, docetaxel, vinblastine, vincristine, vindesine, vinorelbine, colchicine, podophyllotoxin, griseofulvin, or glaziovianin A; and
the proteasome inhibitor is lactacystin, carfilzomib, disulfiram, epigallocatechin-3-gallate, salinosporamide A, oprozomib, delanzomib, epoxomicin, MG132, or β-hydroxy β-methylbutyrate.

8. A method of producing a drug-containing vesicle comprising: mixing a plurality of Janus particles with a drug solution to form a mixed solution; and producing the drug-containing vesicle via a film-hydration method or an injection method;
wherein,
in the film-hydration method, the mixed solution is condensed until a film-like structure is formed, which is then sonicated in a salt solution to produce the drug-containing vesicle;
in the injection method, the mixed solution is injected into the salt solution to produce the drug-containing vesicle;
the drug solution is formed by dissolving a drug in a solvent, which is selected from the group consisting of water, ethanol and dimethyl sulfoxide; and
the drug is an alkylating agent, a nucleoside analogue, a topoisomerase inhibitor, a mitotic inhibitor, a proteasome inhibitor, or an interference RNA.

9. The method of claim 8, further comprising purifying the drug-containing vesicle by dialysis so as to remove any residual non-encapsulated drug.

10. The method of claim 8, wherein the plurality of Janus particles are formed by,
(a) subjecting a carbon source to a heat treatment at a temperature of about 220° C. to about 250° C. until an elastomer is formed;
(b) converting the elastomer into the plurality of Janus particles by treating the elastomer with an alcohol in the presence of a base.

11. The method of claim 10, wherein in the step (a), the carbon source is a mono-glyceride, di-glyceride or a tri-glyceride.

12. The method of claim 11, wherein the carbon source is glyceryl trioleate.

13. The method of claim 10, wherein in the step (b),
the alcohol is selected from the group consisting of methanol, ethanol, propanol, isopropanol 30 and butanol; and
the base is selected from the group consisting of sodium hydroxide, potassium hydroxide, and ammonium hydroxide.

14. The method of claim 8, wherein
the alkylating agent is cyclophosphamide, chlormethine, uramustine, melphalan, chlorambucil, ifosfamide, bendamustine, carmustine, lomustine, streptozocin, cisplatin, carboplatin, dicycloplatin, eptaplatin, lobaplatin, miriplatin, or oxaliplatin;
the nucleoside analogue is didanosine, vidarabine, galidesivir, cytarabine, gemcitabine, emtricitabine, lamivudine, zalcitabine, abacavir, acyclovir, entecavir, stavudine, telbivudine, zidovudine, idoxuridine, or trifluridine;
the topoisomerase inhibitor is amsacrine, etoposide, etoposide phosphate, teniposide, doxorubicin, geni stein, or ICRF-193;
the mitotic inhibitor is paclitaxel, docetaxel, vinblastine, vincristine, vindesine, vinorelbine, colchicine, podophyllotoxin, griseofulvin, or glaziovianin A; and
the proteasome inhibitor is lactacystin, carfilzomib, disulfiram, epigallocatechin-3-gallate, salinosporamide A, oprozomib, delanzomib, epoxomicin, MG132, or β-hydroxy β-methylbutyrate.

15. A method of treating a subject afflicted with a cancer comprising administering to the subject an effective amount of the drug containing vesicles of claim 1.

16. The mothed of claim 15, further comprising irradiating the subject in sequence with a first and a second light respectively having a wavelength of 350-400 nm and 480-550 nm.

17. The method of claim 16, wherein the first light has the wavelength of 385 nm, and the second light has the wavelength of 530 nm.

18. The method of claim 15, wherein the cancer is any of analplastic large cell lymphoma, angiosarcoma, bone cancer, bladder cancer, biliary cancer, brain cancer, breast cancer, cancer of testicles, cancer of connective tissue, cancer of retina, colon cancer, cervical cancer, endometrial cancer, epidermal carcinoma, esophageal squamous cell carcinoma, follicular dentritic cell carcinoma, fallopian tube cancer, gastrointestinal stromal tumor (GIST), glioma, glioblastoma, head and neck cancer, hematopoietic tumors of lymphoid lineage, heptatocellular carcinoma, intestinal cancer, Kaposi's sarcoma, keratoacanthomas, Li-Fraumeni syndrome, lung cancer, malignant ascites, melanoma, mesothelioma, acute myelogenous leukemia (AML), chronic myelogenous leukemia (CML), myelodysplastic syndrome (MDS), myelodysplasia, muscle invasive cancer, nasopharyngeal, neuroendocrine cancer, neuroblastoma, oesophagogastric, ovary cancer, pancreatic cancer, peritoneal cancer, papillary serous mullerian cancer, prostate cancer, prostatic hypertrophy, renal cancer, seminal vesicle tumor, spleen cancer, stomach cancer, small bowel cancer, salivary gland cancer, thyroid cancer, teratcarcinoma, thyroid follicular cancer, leukemia, acute lymphocytic leukemia, chronic lymphocytic leukemia, B-cell lymphoma, Burkitt's lymphoma, multiple myeloma, Hodgkin's lymphoma, Non-Hodgkin's lymphoma, uveal melanoma, uterine sarcoma, Von Hippel-Lindau syndrome (VHL), or Waldenstrom's macroglobulinemia.

19. A method of treating a subject afflicted with a cancer comprising:
administering to the subject an effective amount of a carbon dot liposome (C-dot liposome) formed by a plurality of Janus particles, which are self-assembled into the C-dot liposome, and
irradiating the subject in sequence with a first and a second light respectively having a wavelength of 350-400 nm and 480-550 nm.

20. The method of claim 19, wherein the first light has the wavelength of 385 nm, and the second light has the wavelength of 530 nm.

21. The method of claim 19, wherein the plurality of Janus particles are formed by,
(a) subjecting a carbon source to a heat treatment at a temperature of about 220° C. to about 250° C. until an elastomer is formed;
(b) converting the elastomer into the plurality of Janus particles by treating the elastomer with an alcohol in the presence of a base.

22. The method of claim 21, wherein in the step (a), the carbon source is a mono-glyceride, di-glyceride or a tri-glyceride.

23. The method of claim 22, wherein the carbon source is glyceryl trioleate.

24. The method of claim 21, wherein in the step (b), the alcohol is selected from the group consisting of methanol, ethanol, propanol, isopropanol and butanol; and the base is selected from the group consisting of sodium hydroxide, potassium hydroxide, and ammonium hydroxide.

25. The method of claim 19, wherein the cancer is any of analplastic large cell lymphoma, angiosarcoma, bone cancer, bladder cancer, biliary cancer, brain cancer, breast cancer, cancer of testicles, cancer of connective tissue, cancer of retina, colon cancer, cervical cancer, endometrial cancer, epidermal carcinoma, esophageal squamous cell carcinoma, follicular dentritic cell carcinoma, fallopian tube cancer, gastrointestinal stromal tumor (GIST), glioma, glioblastoma, head and neck cancer, hematopoietic tumors of lymphoid lineage, heptatocellular carcinoma, intestinal cancer, Kaposi's sarcoma, keratoacanthomas, Li-Fraumeni syndrome, lung cancer, malignant ascites, melanoma, mesothelioma, acute myelogenous leukemia (AML), chronic myelogenous leukemia (CML), myelodysplastic syndrome (MDS), myelodysplasia, muscle invasive cancer, nasopharyngeal, neuroendocrine cancer, neuroblastoma, oesophagogastric, ovary cancer, pancreatic cancer, peritoneal cancer, papillary serous mullerian cancer, prostate cancer, prostatic hypertrophy, renal cancer, seminal vesicle tumor, spleen cancer, stomach cancer, small bowel cancer, salivary gland cancer, thyroid cancer, teratcarcinoma, thyroid follicular cancer, leukemia, acute lymphocytic leukemia, chronic lymphocytic leukemia, B-cell lymphoma, Burkitt's lymphoma, multiple myeloma, Hodgkin's lymphoma, Non-Hodgkin's lymphoma, uveal melanoma, uterine sarcoma, Von Hippel-Lindau syndrome (VHL), or Waldenstrom' s macroglobulinemia.
